(19) United States
(12) Patent Application Publication
(10) Pub. No.: US 2026/0217748 A1
(43) Pub. Date: Jul. 30, 2026

(54) IBOGAINE AND NORIBOGAINE PRODRUGS AND METHODS OF USE

(71) Applicant: Atai Therapeutics, Inc., New York, NY (US)

(72) Inventor: Tanweer KHAN, Berlin (DE)

(21) Appl. No.: 19/143,299

(22) PCT Filed: Dec. 28, 2023
(86) PCT No.: PCT/US2023/086216

§ 371 Date: Jun. 25, 2025

Related U.S. Application Data
(60) Provisional application No. 63/477,708, filed on Dec. 29, 2022.

(51) Int. Cl. C07F 9/6561; A61K 31/55; A61K 31/675; C07D 471/18

(52) U.S. Cl. CPC C07F 9/6561; A61K 31/55; A61K 31/675; C07D 471/18

(57) ABSTRACT

Provided herein are compounds of Formula (I), Formula (II), pharmaceutically acceptable salts thereof, and compositions thereof, wherein R, R₁, R₂, and M are defined herein. The disclosed compounds are useful for treating various conditions, including alcoholism, substance abuse disorder, and opioid use disorder.

### CROSS-REFERENCE TO RELATED APPLICATION

This application claims the benefit of priority to U.S. Provisional Application No. 63/477,708, filed Dec. 29, 2022, which is hereby incorporated by reference in its entirety for all purposes.

### BACKGROUND OF THE INVENTION

Ibogaine is a naturally occurring psychoactive compound with therapeutic uses for the treatment of substance use disorders. In particular, a recent animal research showed that ibogaine reduced self-administration of several drugs, including opiates, cocaine, and ethanol (Belgers et al., Transl Psychiatry, 31; 6(5):e826, 2016). Ibogaine is metabolized in the body to its main metabolite, noribogaine, which is a non-hallucinogenic compound with an overlapping, but distinct profile of pharmacological effects.

The pharmacological basis for the therapeutic effects of ibogaine and noribogaine are unclear. Despite these benefits, the pharmacokinetic and biodistribution profile of ibogaine potentially limits its therapeutic utility.

There is a need for prodrugs of ibogaine and noribogaine. The present disclosure addresses these needs.

### BRIEF SUMMARY OF THE INVENTION

In one aspect, the present disclosure provides a compound of Formula (I):

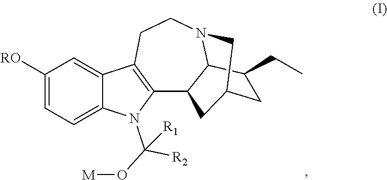

or a pharmaceutically acceptable salt thereof, wherein:

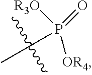

M is hydrogen, C₁-C₈ alkyl, —CR₁R₂—O—C₁-C₈ alkyl, or
R is hydrogen, C₁-C₈ alkyl, or cycloalkyl;
R₁ and R₂ are independently hydrogen, C₁-C₈ alkyl, or cycloalkyl, or R₁ and R₂ together with the atoms to which they are attached form a C₃-C₈ cycloalkyl; and
R₃ and R₄ are independently hydrogen, C₁-C₈ alkyl, cycloalkyl, alkylene-cycloalkyl, aryl, or R₃ and R₄ together with the atoms to which they are attached form a heterocycle, wherein when R is methyl and R₁ and R₂ are hydrogen, M is not hydrogen.

In one aspect, the present disclosure provides a compound of Formula (II):

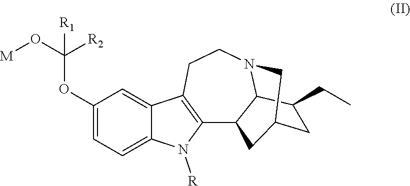

or a pharmaceutically acceptable salt thereof wherein:

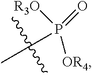

M is hydrogen, C₁-C₈ alkyl or
R is hydrogen, C₁-C₈ alkyl, or cycloalkyl;
R₁ and R₂ are independently hydrogen, C₁-C₈ alkyl, or cycloalkyl, or R₁ and R₂ together with the atoms to which they are attached form a C₃-C₈ carbocycle; and
R₃ and R₄ are independently hydrogen, C₁-C₈ alkyl, cycloalkyl, alkylene-cycloalkyl, aryl, or R₃ and R₄ together with the atoms to which they are attached form a heterocycle.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “treating” as used herein with regard to a patient or subject, refers to improving at least one symptom of the patient's or subject's disorder. In embodiments, treating can be improving, or at least partially ameliorating a disorder or one or more symptoms of a disorder.

The term “therapeutically effective” applied to dose or amount refers to that quantity of a compound or pharmaceutical formulation that is sufficient to result in a desired clinical benefit after administration to a patient or subject in need thereof.

The terin “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammnonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁-5, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising tip to 12 carbon atoms is a C₁-C₁₂ alkyl, an alkyl comprising tip to 10 carbon atoms is a C₁-C₁₀ alkyl, an alkyl comprising up to 6 carbon atoms is a C₁-C₆ alkyl and an alkyl comprising tip to 5 carbon atoms is a C₁-C₈ alkyl. A C₁-C₈ alkyl includes C₅ alkyls, C₄ alkyls, C₃ alkyls, C₂ alkyls and C₁ alkyl (i.e., methyl). A C₁-C₆ alkyl includes all moieties described above for C₁-C₈ alkyls but also includes C₆ alkyls. A C₁-C₁₀ alkyl includes all moieties described above for C₁-C₈ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, irnidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, including compounds with aromatic resonance structures (e.g., 2-pyridone), and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzofuranyl, benzooxazolyl, benzothiadiazolyl, benzo[b][1,4]dioxepinyl, 1,4 benzodioxanyl, benzonaphthofuranyl, benzoxazolyl, benzodioxolyl, benzodioxinyl, benzopyranyl, benzopyranonyl, benzofuranonyl, benzothienyl (benzothiophenyl), benzotriazolyl, benzo[4,6]imidazo[1,2 a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2 oxoazepinyl, oxazolyl, oxiranyl, 1-oxidopyridinyl, 1 oxidopyrimidinyl, -oxidopyrazinyl, 1-oxidopyridazinyl, 1 phenyl 11-1 pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

The term “substituted” used herein means any of the groups described herein (e.g, alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O—))Rₕ, —NR_{g}C(═O)NRgRh, —NR_{g}C(═O))ORₕ, —NR_{g}SO₂Rₕ, —OC(═O)NR_{g} Rₕ, —OR_{g}, —SR_{g}, —SOR_{g}, —SO₂R, —OSO₂R, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkyl, haloalkyl, haloalkenyl, haloalkynyl, heterocyclyl, A-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkyl, haloalkyl, haloalkenyl, haloalkynyl, heterocyclyl, N-heterocyclyl, heterocyclylalkyl, heteroaryl, N-heteroaryl and/or heteroarylalkyl group. In some embodiments, “substituted” further means any alkyl, cycloalkyl or heterocyclylalkyl in which one or more hydrogen atoms is replaced by an isotope e.g., deuterium. In addition, each of the foregoing substituents can also be optionally substituted with one or more of the above substituents.

### Compounds

The present disclosure provides compounds that are prodrugs or analogs of ibogaine and noribogaine as well as pharmaceutical compositions thereof and uses thereof in treating various diseases and disorders.

In embodiments, the present disclosure provides a compound of Formula (I),

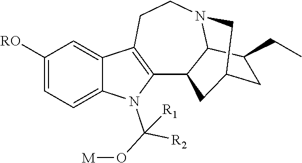

or a pharmaceutically acceptable salt thereof, wherein:
M is hydrogen, C₁-C₈ alkyl, —CR₁R₂—O—C₁-C₈ alkyl, or

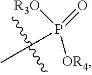

R is hydrogen, C₁-C₈ alkyl, or cycloalkyl;
R₁ and R₂ are independently hydrogen, C₁-C₈ alkyl, or cycloalkyl, or R₁ and R₂ together with the atoms to which they are attached form a C₃-C₈ cycloalkyl; and
R₃ and R₄ are independently hydrogen, C₁-C₈ alkyl, cycloalkyl, alkylene-cycloalkyl, aryl, or R₃ and R₄ together with the atoms to which they are attached form a heterocycle, wherein when R is methyl and R₁ and R₂ are hydrogen, M is not hydrogen.

In embodiments, M is hydrogen.

In embodiments, M is C₁-C₈ alkyl. In embodiments, M is C₁-C₃ alkyl. In embodiments, M is methyl. In embodiments, NI is ethyl.

In embodiments, M is —CR₁R₂—O—C₁-C₈ alkyl. In embodiments, at least one of R₁ and R₂ is hydrogen. In embodiments, R₁ and R₂ are hydrogen. In embodiments, at least one of R₁ and R₂ is C₁-C₈alkyl. In embodiments, R₁ and R₂ are C₁-C₈ alkyl. In embodiments, at least one of R₁ and R₂ is C₁-C₃ alkyl. In embodiments, R₁ and R₂ are C₁-C₃ alkyl. In embodiments, R₁ is C₁-C₈ alkyl and R₂ is hydrogen. In embodiments, R₁ is C₁-C₃ alkyl and R₂ is hydrogen. In embodiments, at least one of R₁ and R₂ is cycloalkyl. In embodiments, R₁ and R₂ are cycloalkyl.

In embodiments, M is —CH₂—O—CH₃. In embodiments, M is —CH(CH₃)—O—CH₃.

In embodiments, R₁ and R₂ together with the atoms to which they are attached form a C₃-C₈ cycloalkyl.

In embodiments, at least one of R₁ and R₂ is a substituted C₁-C₈ alkyl. In embodiments, one or more hydrogen atoms of C₁-C₈ alkyl of Rⱼ or R₂ are replaced with oxygen, carbonyl, carboxyl, ester, ether, amide, imine, oxime, hydrazone, nitrile, is a carbonyl, an amide, and an ether. In embodiments, at least one of R₁ and R₂ is a C₁-C₈ alkyl the substituent of C₁-C₈ alkyl is substituted with one or more of —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. In embodiments, R_{g} and Rₕ are the same or different and independently hydrogen, alkyl, alkenyl, alkoxy, alkylamino, thioalkyl, aryl, cycloalkyl, cycloalkenyl, cycloalkyl, haloalkyl, haloalkenyl, heterocyclyl, heteroaryl, and/or heteroarylalkyl.

In embodiments, M is

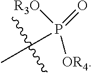

In embodiments, at least one of R₃ and R₄ is hydrogen. In embodiments, R₃ and R₄ are hydrogen. In embodiments, at least one of R₃ and R₄ is C₁-C₈ alkyl. In embodiments, R₃ and R₄ are C₁-C₈ alkyl. In embodiments, at least one of R₃ and R₄ is C₁-C₃ alkyl. In embodiments, R₃ and R₄ are C₁-C₃ alkyl. In embodiments, R₃ and R₄ are methyl. In embodiments, R₃ and R₄ are ethyl. In embodiments, at least one of R₃ and R₄ is cycloalkyl. In embodiments, at least one of R₃ and R₄ is alkylene-cycloalkyl. In embodiments, at least one of R₃ and R₄ is aryl. In embodiments, R₃ and R₄ together with the atoms to which they are attached form a 5- to 8-membered heterocycle.

In embodiments, R is hydrogen. In embodiments, R is C₁-C₈ alkyl. In embodiments, R is C₁-C₃ alkyl. In embodiments, R is methyl. In embodiments, R is cycloalkyl.

In embodiments, when R is methyl and R₁ and R₂ are hydrogen, M is not hydrogen.

In embodiments, the present disclosure provides a compound shown in Table 1, or a pharmaceutically acceptable salt thereof

TABLE 1
Compounds

| Compound | | |
|---|---|---|
| No. | Structure | Name |
| 1 | 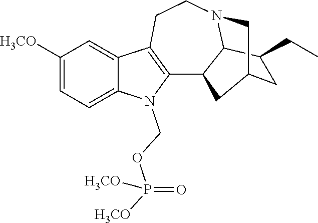 [SMILES: CC[C@H]1CC2C[C@H]3c4c(c5cc(OC)ccc5n4COP(=O)(OC)OC)CC[N@@](C2)C13] | ((6R,7S,11S)-7-ethyl-2-methoxy- 6,6a,7,8,9,10,12,13-octahydro-5H-6,9- methanopyrido[1′,2′:1,2]azepino[4,5- b]indol-5-yl)methyl dimethyl phosphate |
| 2 | 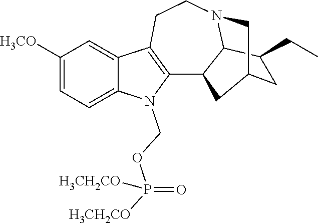 | diethyl (((6R,7S,11S)-7-ethyl-2-methoxy- 6,6a,7,8,9,10,12,13-octahydro-5H-6,9- methanopyrido[1′,2′:1,2]azepino[4,5- b]indol-5-yl)methyl) phosphate |
| 3 | 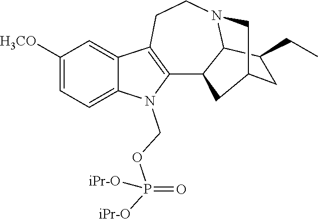 | ((6R,7S,11S)-7-ethyl-2-methoxy- 6,6a,7,8,9,10,12,13-octahydro-5H-6,9- methanopyrido[1′,2′:1,2]azepino[4,5- b]indol-5-yl)methyl diisopropyl phosphate |
| 4 | 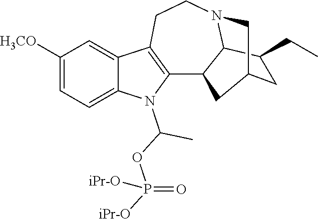 | 1-((6R,7S,11S)-7-ethyl-2-methoxy- 6,6a,7,8,9,10,12,13-octahydro-5H-6,9- methanopyrido[1′,2′:1,2]azepino[4,5- b]indol-5-yl)ethyl diisopropyl phosphate |
| 5 | 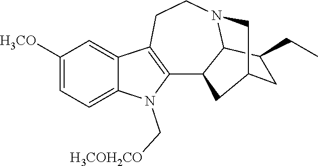 | (6R,7S,11S)-7-ethyl-2-methoxy-5- ((methoxymethoxy)methyl)- 6,6a,7,8,9,10,12,13-octahydro-5H-6,9- methanopyrido[1′,2′:1,2]azepino[4,5- b]indole |
| 6 | 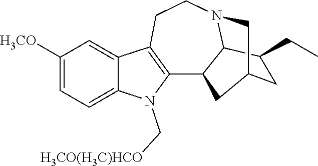 | (6R,7S,11S)-7-ethyl-2-methoxy-5-((2- methoxy-2l5-ethoxy)methyl)- 6,6a,7,8,9,10,12,13-octahydro-5H-6,9- methanopyrido[1′,2′:1,2]azepino[4,5- b]indole |

In embodiments, the present disclosure provides a compound of Formula (II):

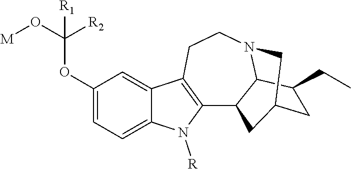

or a pharmaceutically acceptable salt thereof, wherein:
M is hydrogen, C₁-C₈ alkyl or

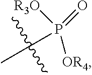

R is hydrogen, C₁-C₈ alkyl, or cycloalkyl;
R₁ and R₂ are independently hydrogen, C₁-C₈ alkyl, or cycloalkyl, or R₁ and R₂ together with the atoms to which they are attached form a C₃-C₈ carbocycle; and
R₃ and R₄ are independently hydrogen, C₁-C₈ alkyl, cycloalkyl, alkylene-cycloalkyl, aryl, or R₃ and R₄ together with the atoms to which they are attached form a heterocycle.

In embodiments, M is hydrogen.

In embodiments, M is C₁-C₈ alkyl. In embodiments, M is C₁-C₃ alkyl. In embodiments, M is methyl. In embodiments, M is ethyl.

In embodiments, M is

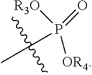

In embodiments, at least one of R₃ and R₃ is hydrogen. In embodiments, R₃ and R₄ are hydrogen. In embodiments, at least one of R₃ and R₄ is C₁-C₈ alkyl. In embodiments, R and R₄ are C₁-C₅ alkyl. In embodiments, at least one of R₃ and R₄ is C₁-C₃ alkyl. In embodiments, R₃ and R₄ are C₁-C₃ alkyl. In embodiments, R₃ and R₄ are methyl. In embodiments, R₃ and R₄ are ethyl. In embodiments, at least one of R₃ and R₄ is cycloalkyl. In embodiments, at least one of R₃ and R₄ is alkylene-cycloalkyl. In embodiments, at least one of R₃ and R₄ is aryl. In embodiments, R₃ and R₄ together with the atoms to which they are attached form a 5- to 8-membered heterocycle.

In embodiments, R is hydrogen. In embodiments, R is C₁-C₈ alkyl. In embodiments, R is methyl. In embodiments, R is cycloalkyl.

In embodiments, R₁ and R₂ are hydrogen. In embodiments, R₁ is C₁-C₈ alkyl and R₂ is hydrogen. In embodiments, R₁ is C₁-C₃ alkyl and R₂ is hydrogen. In embodiments, at least one of R₁ and R₂ is hydrogen. In embodiments, R₁ and R₂ are hydrogen. In embodiments, at least one of R₁ and R₂ is C₁-C₈ alkyl. In embodiments, R₁ and R₂ are C₁-C₈ alkyl. In embodiments, at least one of R₁ and R₂ is C₁-C₃ alkyl. In embodiments, R₁ and R₂ are C₁-C₃ alkyl. In embodiments, R₁ and R₂ are methyl. In embodiments, R₁ and R₂ are ethyl. In embodiments, R₁ is C₁-C₈ alkyl and R₂ is hydrogen. In embodiments, R₁ is C₁-C₃ alkyl and R₂ is hydrogen. In embodiments, at least one of R₁ and R₂ is cycloalkyl. In embodiments, R₁ and R₂ are cycloalkyl.

In embodiments, R₁ and R₂ together with the atoms to which they are attached form a C₃-C₈ cycloalkyl.

In embodiments, at least one of R₁ and R₂ is a substituted C₁-C₈ alkyl. In embodiments, one or more hydrogen atoms of C₁-C₈ alkyl of R₁ or R₂ are replaced with oxygen, carbonyl, carboxyl, ester, ether, amide, imine, oxime, hydrazone, nitrile, is a carbonyl, an amide, and an ether. In embodiments, at least one of R₁ and R₂ is a C₁-C₈ alkyl the substituent of C₁-C₈ alkyl is substituted with one or more of —NR_{g}Rₕ, —NR_{g}C(═O)Rₕ, —NR_{g}C(═O)NR_{g}Rₕ, —NR_{B}C(═O)ORₕ, —NR_{g}SO₂R, —OC(═O)NR_{g}Rₕ, OR_{g}, SR_{g}, SOR_{g}, —SO₂R_{g}, —OSO₂R_{g}, —SO₂OR_{g}, ═NSO₂R_{g}, and —SO₂NR_{g}Rₕ In embodiments, R_{g} and Rₕ are the same or different and independently hydrogen, alkyl, alkenyl, alkoxy, alkylamino, thioalkyl, aryl, cycloalkyl, cycloalkenyl, cycloalkyl, haloalkyl, haloalkenyl, heterocyclyl, heteroaryl, and/or heteroarylalkyl.

In embodiments, the present disclosure provides a compound shown in Table 2, or a pharmaceutically acceptable salt thereof.

TABLE 2
Compounds

| Compound | | |
|---|---|---|
| No. | Structure | Name |
| 7 | 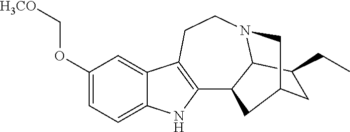 [SMILES: CC[C@H]1CC2C[C@H]3c4[nH]c5ccc(OCOC)cc5c4CC[N@@](C2)C13] | (6R,7S,11S)-7-ethyl-2- (methoxymethoxy)- 6,6a,7,8,9,10,12,13- octahydro-5H-6,9- methanopyrido[1′,2′:1,2]azepi- no[4,5-b]indole |
| 8 | 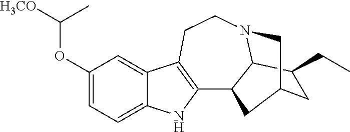 [SMILES: CC[C@H]1CC2C[C@H]3c4[nH]c5ccc(OC(C)OC)cc5c4CC[N@@](C2)C13] | (6R,7S,11S)-7-ethyl-2-(1- methoxyethoxy)- 6,6a,7,8,9,10,12,13- octahydro-5H-6,9- methanopyrido[1′,2′:1,2]azepi- no[4,5-b]indole |
| 9 | 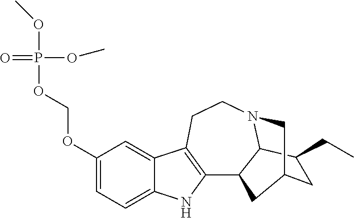 [SMILES: CC[C@H]1CC2C[C@H]3c4[nH]c5ccc(OCOP(=O)(OC)OC)cc5c4CC[N@@](C2)C13] | (((6R,7S,11S)-7-ethyl- 6,6a,7,8,9,10,12,13- octahydro-5H-6,9- methanopyrido[1′,2′:1,2]azepi- no[4,5-b]indol-2- yl)oxy)methyl dimethyl phosphate |
| 10 | 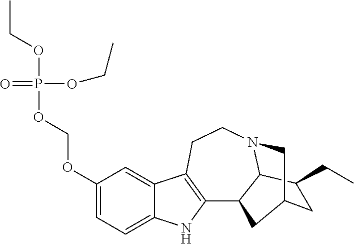 [SMILES: CCOP(=O)(OCC)OCOc1ccc2[nH]c3c(c2c1)CC[N@@]1CC2C[C@H](CC)C1[C@H]3C2] | diethyl ((((6R,7S,11S)-7- ethyl-6,6a,7,8,9,10,12,13- octahydro-5H-6,9- methanopyrido[1′,2′:1,2]azepi- no[4,5-b]indol-2- yl)oxy)methyl)phosphate |
| 11 | 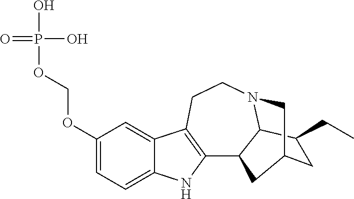 [SMILES: CC[C@H]1CC2C[C@H]3c4[nH]c5ccc(OCOP(=O)(O)O)cc5c4CC[N@@](C2)C13] | (((6R,7S,11S)-7-ethyl- 6,6a,7,8,9,10,12,13- octahydro-5H-6,9- methanopyrido[1′,2′:1,2]azepi- no[4,5-b]indol-2- yl)oxy)methyl dihydrogen phosphate |

### Compositions

The present disclosure provides pharmaceutical compositions for treating various conditions or disorders in a subject in need thereof. In some embodiments, a pharmaceutical composition comprises one or more compounds of the present disclosure (e.g., a compound of Formula (I), Formula (H), Table 1, or Table 2, or pharmaceutically acceptable salts thereof. In embodiments, the pharmaceutical compositions comprise pharmaceutically acceptable excipients and adjuvants.

The pharmaceutically acceptable excipients and adjuvants are added to the composition or formulation for a variety of purposes. In embodiments, a pharmaceutical composition comprising one or more compounds disclosed herein, or a pharmaceutically acceptable salt thereof, further comprise a pharmaceutically acceptable carrier. In embodiments, a pharmaceutically acceptable carrier includes a pharmaceutically acceptable excipient, binder, and/or diluent. In embodiments, suitable pharmaceutically acceptable carriers include, but are not limited to, inert solid fillers or diluents and sterile aqueous or organic solutions. In embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

### Methods of Treatment

In embodiments, the present disclosure provides methods of treating various conditions by administering a pharmaceutical composition comprising one or more compounds of the present disclosure and a pharmaceutically acceptable excipient to a subject in need thereof.

In embodiments, the condition is alcoholism, substance abuse disorder, or opioid use disorder. In embodiments, the condition is opioid use disorder. In embodiments, the condition is symptoms of detoxification and/or withdrawal that result from stopping or reducing the use of a medication or drug. In embodiments, the medication or drug is a substance with a high potential for dependency or abuse. In embodiments, the condition is related to compulsive/repetitive behaviors, underlying neurocircuitries and neuroplastic effects (e.g., addictions such as gambling or sex, eating disorders, obsessive compulsive disorder (OCD), major depressive disorder (MDD), treatment-resistant depression (TRD), anxiety, post-traumatic stress disorder) (PTSD), attention-deficit/hyperactivity disorder (ADHD), autism spectrum disorder (ASD), and the like).

In embodiments, the present disclosure provides methods of treating substance abuse disorder in a subject in need thereof, comprising administering a therapeutically effective amount of a compound disclosed herein (e.g., compounds of Formula (I) or Formula (II) or compounds of Table 1 or Table 2), a pharmaceutically acceptable salt thereof, or a pharmaceutical composition thereof to the subject.

In embodiments, the present disclosure provides methods of treating opioid use disorder in a subject in need thereof, comprising administering a therapeutically effective amount of a compound disclosed herein (e.g., compounds of Formula (I) or Formula (II) or compounds of Table 1 or Table 2), a pharmaceutically acceptable salt thereof, or a pharmaceutical composition thereof to the subject.

### Numbered Embodiments

1. A compound of Formula (I)

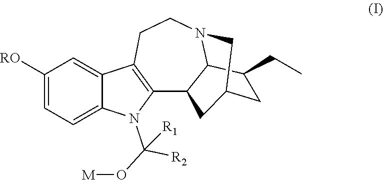

or a pharmaceutically acceptable salt thereof, wherein:
M is hydrogen, C₁-C₈ alkyl, —CR₁R₂—O—C₁-C₈ alkyl, or

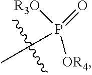

R is hydrogen, C₁-C₈ alkyl, or cycloalkyl;
R₁ and R₂ are independently hydrogen, C₁-C₅ alkyl, or cycloalkyl, or R₁ and R₂ together with the atoms to which they are attached form a C₃-C₈ cycloalkyl; and
R₃ and R₄ are independently hydrogen, C₁-C₈ alkyl, cycloalkyl, alkylene-cycloalkyl, aryl, or R₃ and R₄ together with the atoms to which they are attached form a heterocycle, wherein when R is methyl and R₁ and R₂ are hydrogen, M is not hydrogen.
2. The compound of embodiment 1, wherein M is C₁-C₈ alkyl.
3. The compound of embodiment 1, wherein M is C₁-C₃ alkyl.
4. The compound of embodiment 1, wherein M is

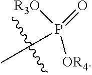

5. The compound of embodiment 1, wherein M is —CR₁R₂—O—C₁-C₈ alkyl.
6. The compound of embodiment 5, wherein M is —CH₂—O—CH₃.
7. The compound of embodiment 5, wherein M is —CH(CH₃)—O—CH₃.
8. The compound of embodiment 4, wherein R₃ and R₄ are independently C₁-C₈ alkyl.
9. The compound of embodiment 4, wherein R₁ and R₄ are independently C₁-C₃ alkyl.
10. The compound of embodiment 4, wherein R₃ and R₄ together with the atoms to which they are attached form a 5- to 8-membered heterocycle.
11. The compound of any one of embodiments 1-10, wherein R₁ and R₂ are hydrogen.
12. The compound of any one of embodiments 1-10, wherein R₁ is C₁-C₈ alkyl and R₂ is hydrogen.
13. The compound of any one of embodiments 1-10, wherein R₁ is C₁-C₃ alkyl and R₂ is hydrogen.
14. The compound of any one of embodiments 1-10, wherein at least one of R₁ and R₂ is a substituted C₁-C₈ alkyl.
15. The compound of any one of embodiments 1-14, wherein R is hydrogen.
16. The compound of any one of embodiments 1-14, wherein R is C₁-C₈ alkyl.
17. The compound of any one of embodiments 1-14, wherein R is methyl.
18. The compound of embodiment 1, having the formula:

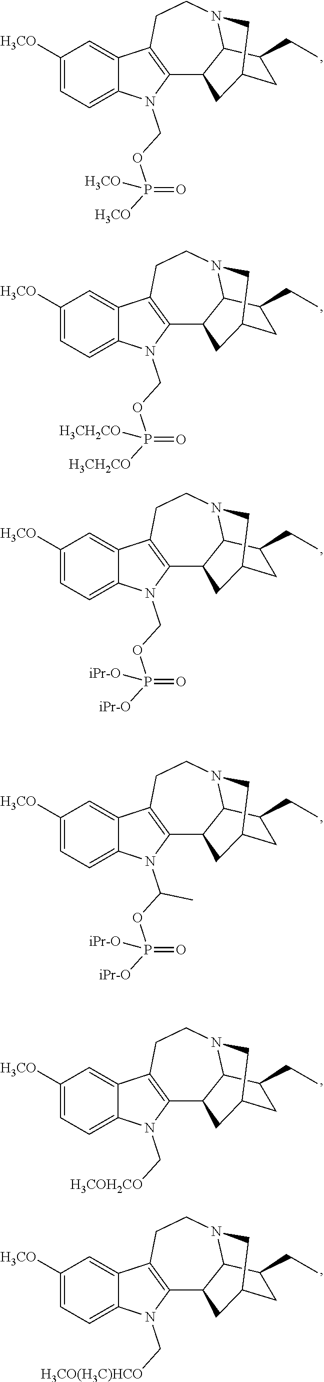

or a pharmaceutically acceptable salt thereof.
19. A compound of Formula (II):

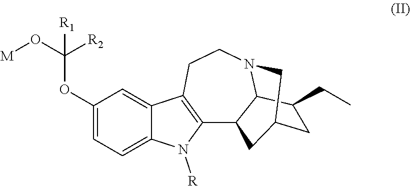

or a pharmaceutically acceptable salt thereof, wherein:

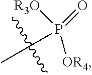

M is hydrogen, C₁-C₈ alkyl or R₄
R is hydrogen, C₁-C₈ alkyl, or cycloalkyl;
R₁ and R₂ are independently hydrogen, C₁-C₈ alkyl, or cycloalkyl, or R₁ and R₂ together with the atoms to which they are attached form a C₃-C₈ carbocycle; and
R₃ and R₄ are independently hydrogen, C₁-C₈ alkyl, cycloalkyl, alkylene-cycloalkyl, aryl, or R₃ and R₄ together with the atoms to which they are attached form a heterocycle.
20. The compound of embodiment 19, wherein M is C₁-C₈ alkyl.
21. The compound of embodiment 19, wherein M is C₁-C₃ alkyl.

22. The compound of any embodiment 19, wherein M is

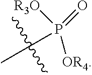

23. The compound of embodiment 22, wherein R₃ and R₄ are independently C₁-C₈ alkyl.
24. The compound of embodiment 22, wherein R₃ and R₄ are independently C₁-C₃ alkyl.
25. The compound of embodiment 19, wherein R₃ and R₄ together with the atoms to which they are attached form a 5- to 8-membered heterocycle.
26. The compound of any one of embodiments 19-25, wherein R₁ and R₂ are hydrogen.
27. The compound of any one of embodiments 19-25, wherein R₁ is C₁-C₈ alkyl and R₂ is hydrogen.
28. The compound of any one of embodiments 19-25, wherein R₁ is C₁-C₃ alkyl and R₂ is hydrogen.
29. The compound of any one of embodiments 19-28, wherein R is hydrogen.
30. The compound of any one of embodiments 19-28, wherein R is C₁-C₈ alkyl.
31. The compound of any one of embodiments 19-28, wherein R is methyl.
32. The compound of embodiment 19, having the formula:

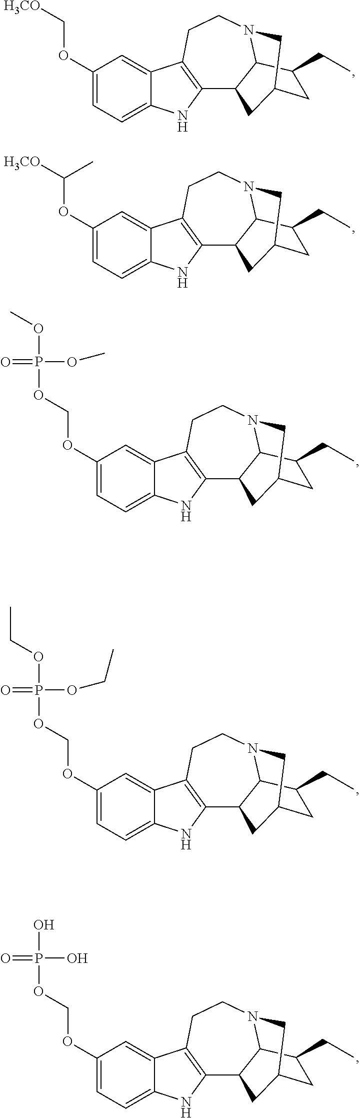 [SMILES: CC[C@H]1CC2C[C@H]3c4[nH]c5ccc(OCOC)cc5c4CC[N@@](C2)C13 ; CC[C@H]1CC2C[C@H]3c4[nH]c5ccc(OC(C)OC)cc5c4CC[N@@](C2)C13 ; CC[C@H]1CC2C[C@H]3c4[nH]c5ccc(OCOP(=O)(OC)OC)cc5c4CC[N@@](C2)C13 ; CCOP(=O)(OCC)OCOc1ccc2[nH]c3c(c2c1)CC[N@@]1CC2C[C@H](CC)C1[C@H]3C2 ; CC[C@H]1CC2C[C@H]3c4[nH]c5ccc(OCOP(=O)(O)O)cc5c4CC[N@@](C2)C13]

or a pharmaceutically acceptable salt thereof.
33. A pharmaceutical composition, comprising a compound of any one of embodiments 1-32 and a pharmaceutically acceptable excipient.

### EXAMPLES

The following schemes provide methods for preparing the compounds of the present disclosure.

### Example 1. General Synthesis Scheme

The compounds of the present disclosure may be prepared according to Scheme 1.

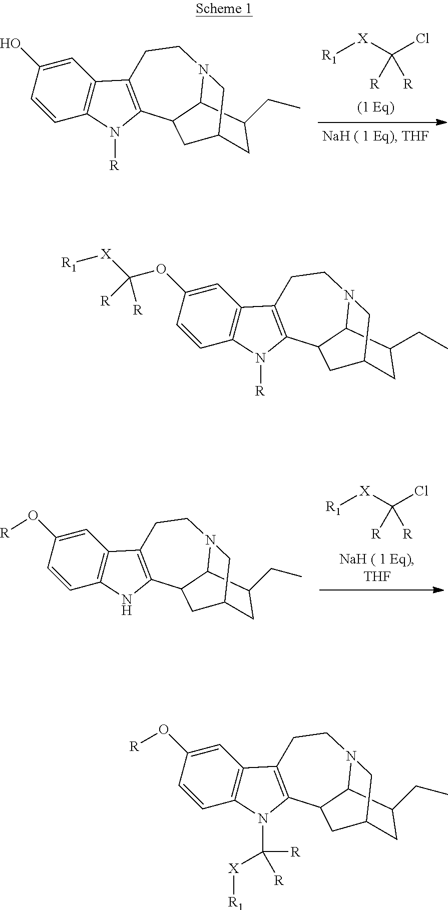

### Example 2. Synthesis of diethyl (((6R,7S,11S)-7-ethyl-2-methoxy-6,6a,7,8,9,10,12,13-octahydro-5H-6,9-methanopyrido[1′, 2′: 1,2]azepino[4,5-b]indol-5-yl)methyl) phosphate (Compound 2)

The Compound 2 was synthesized from ibogaine according to Scheme 2.

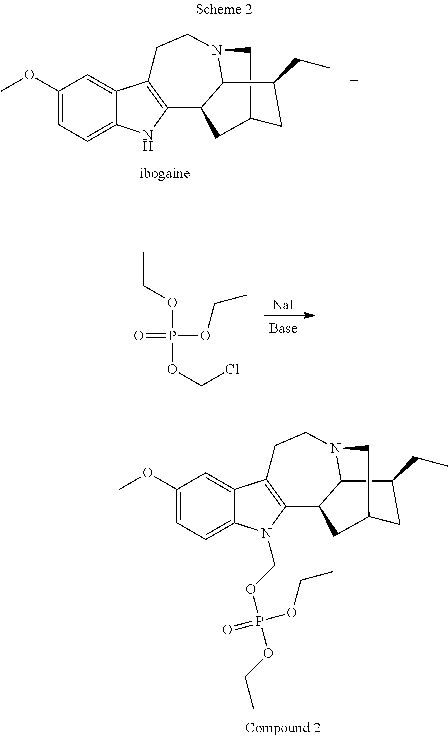

### Example 3. Synthesis of diethyl ((((6R,7S,11S)-7-ethyl-6,6a,7,8,9,10,12,13-octahydro-5H-6,9-methanopyrido[1′,2′:1,2]azepino[4,5-b]indol-2-yl)oxy)methyl) phosphate (Compound 10)

Compound 10 was synthesized from noribogaine according to Scheme 3.

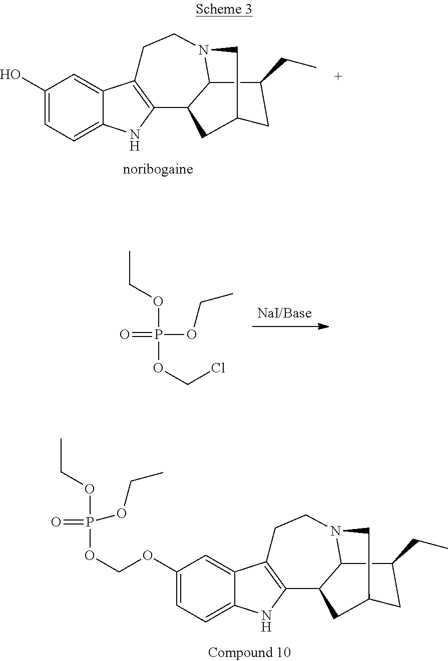 [SMILES: CC[C@H]1CC2C[C@H]3c4[nH]c5ccc(O)cc5c4CC[N@@](C2)C13 ; CCOP(=O)(OCC)OCCl ; CCOP(=O)(OCC)OCOc1ccc2[nH]c3c(c2c1)CC[N@@]1CC2C[C@H](CC)C1[C@H]3C2]

### Example 4: (6R,7S,9S,11S)-7-ethyl-2-(1-methoxyethoxy)-6,6a,7,8,9,10,12,13-octahydro-5H-6,9-methanopyrido[1′,2′:1,2]azepino[4,5-b]indole (3)

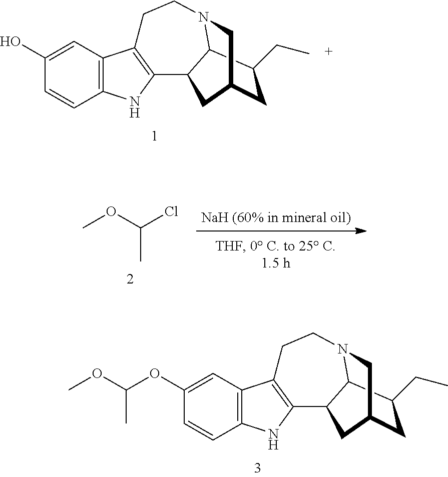

Under an inert atmosphere of argon gas, sodium hydride (12 mg, 337 μmol, 60% in mineral oil) was dissolved in THF (2 mL) and cooled to −5° C. before Noribogaine (Compound 1, 100 mg, 337 μmol), dissolved in THF (1 mL), was added, and stirred for 15 minutes. Next, 1-chloro-1-methoxyethane (31 mg, 337 μmol), diluted in THF (1 mL), was added dropwise over 15 minutes. The reaction was warmed and maintained at 25° C. for an hour. The reaction mixture was cooled to 0° C., quenched with (2 ml) of methanol and concentrated over reduced pressure. The residue was diluted with ethyl acetate (100 mL), and the organic phase was washed with brine (40 mL×3). The organic phase was dried over sodium sulfate, filtered and the solvent was evaporated off. The crude reaction mixture was purified by normal phase silica gel column chromatography, using 0% to 15% MeOH in DCM as mobile phase. The product-containing fractions were combined and dried under reduced pressure to afford the desired product (Compound 3) (74 mg, 62% yield). ¹H NMR (400 MHz, CD₃CN) S 8.82 (br s, 1H), 7.18 (d, J=8.6 Hz, 1H), 7.04 (d, J=2.3 Hz, 1H), 6.78 (dd, 1=8.6, 2.3 Hz, 1H), 5.26 (q, J=5.2 Hz, 1H), 3.40 (s, 3H), 3.38-3.23 (m, 2H), 3.17-3.06 (m, 2H), 3.06-2.95 (m, 2H), 2.91-2.85 (m, 1H), 2.68-2.58 (m, 1H), 2.15-2.05 (m, 1H), 1.91-1.82 (m, 2H), 1.64-1.57 (m, 2H), 1.54-1.48 (m, 1H), 1.43 (d, J=5.2 Hz, 3H), 1.29 (s, 1H), 1.23-1.15 (m, 1H), 0.92 (t, J=7.2 Hz, 3H). ESI-MS: measured m/z 355.33 [M+H]⁺. Purity by HPLC: 98.1% at 254 nm.

The functional characteristics of Compound 3 are summarized in Table 3.

TABLE 3
Characterization of Compound 3

| Assay | Compound 3 |
|---|---|
| SERT Cellular Functional assay IC₅₀ (μM) | 11.9 |
| SERT Binding assay IC₅₀ (μM) | NI |
| KOR1/KOR2 Binding assay IC₅₀ (μM) | NI |
| Chrom logD | 4.9 |
| Microsome half-life RLM +NADPH/−NADPH (min.) | 4/>200 |
| PBS solubility μM | 245 |
| hERG Binding assay IC₅₀ (μM) | >10 |
| Noribogaine Release, SGF, 1 hour, 37° C. (%) | 61 |
| Noribogaine Release, SIF, 1 hour, 37° C. (%) | 55 |

NI: No detectable inhibition

1. A compound of Formula (I): 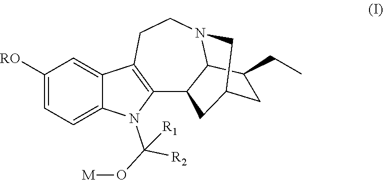
or a pharmaceutically acceptable salt thereof, wherein:
M is hydrogen, C₁-C₈ alkyl, —CR₁R₂—O—C₁-C₅ alkyl, or 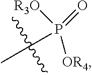
R is hydrogen, C₁-C₈ alkyl, or cycloalkyl;
R₁ and R₂ are independently hydrogen, C₁-C₈ alkyl, or cycloalkyl, or R₁ and R₂ together with the atoms to which they are attached form a C₃-C₈ cycloalkyl; and
R₃ and R₄ are independently hydrogen, C₁-C₈ alkyl, cycloalkyl, alkylene-cycloalkyl, aryl, or R₃ and R₄ together with the atoms to which they are attached form a heterocycle,
wherein when R is methyl and R₁ and R₂ are hydrogen, M is not hydrogen.

2. The compound of claim 1, wherein M is C₁-C₈ alkyl.

3. The compound of claim 1, wherein M is C₁-C₃ alkyl.

4. The compound of claim 1, wherein M is

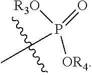

5. The compound of claim 1, wherein M is —CR₁R₂—O—C₁-C₈ alkyl.

6. The compound of claim 1, wherein M is —CH₂—O—CH₃.

7. The compound of claim 1, wherein M is —CH(CH₃)—O—CH₃.

8. The compound of claim 4, wherein R₃ and R₄ are independently C₁-C₈ alkyl.

9. The compound of claim 4, wherein R₃ and R₄ are independently C₁-C₃ alkyl.

10. The compound of claim 4, wherein R₃ and R₄ together with the atoms to which they are attached form a 5- to 8-membered heterocycle.

11. The compound of any one of claims 1-10, wherein R₁ and R₂ are hydrogen.

12. The compound of any one of claims 1-10, wherein R₁ is C₁-C₈ alkyl and R₂ is hydrogen.

13. The compound of any one of claims 1-10, wherein R₁ is C₁-C₃ alkyl and R₂ is hydrogen.

14. The compound of any one of claims 1-10, wherein at least one of R₁ and R₂ is a substituted C₁-C₈ alkyl.

15. The compound of any one of claims 1-14, wherein R is hydrogen.

16. The compound of any one of claims 1-14, wherein R is C₁-C₅ alkyl.

17. The compound of any one of claims 1-14, wherein R is methyl.

18. The compound of claim 1, having the formula: 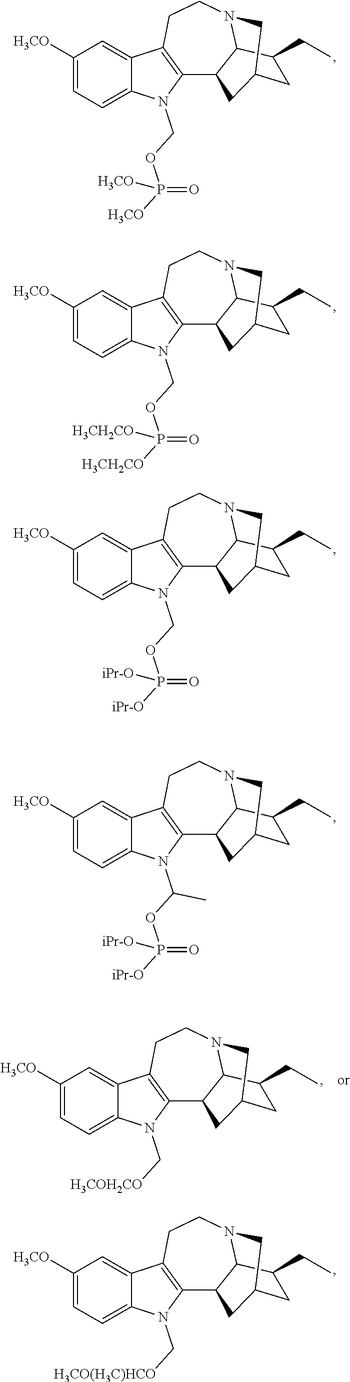
or a pharmaceutically acceptable salt thereof.

19. A compound of Formula (II): 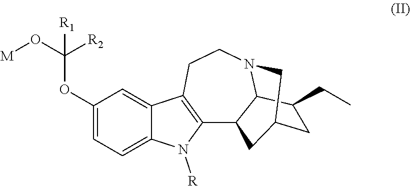
or a pharmaceutically acceptable salt thereof, wherein:
M is hydrogen, C₁-C₈ alkyl or 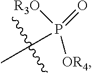
R is hydrogen, C₁-C₈alkyl, or cycloalkyl;
R₁ and R₂ are independently hydrogen, C₁-C₈ alkyl, or cycloalkyl, or R₁ and R₂ together with the atoms to which they are attached form a C₃-C₈ carbocycle; and
R₃ and R₄ are independently hydrogen, C₁-C₈ alkyl, cycloalkyl, alkylene-cycloalkyl, aryl, or R₃ and R₄ together with the atoms to which they are attached form a heterocycle.

20. The compound of claim 19, wherein M is C₁-C₈ alkyl.

21. The compound of claim 19, wherein M is C₁-C₃ alkyl.

22. The compound of any claim 19, wherein M is

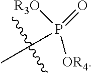

23. The compound of claim 22, wherein R₃ and R₄ are independently C₁-C₈ alkyl.

24. The compound of claim 22, wherein R₃ and R₄ are independently C₁-C₃ alkyl.

25. The compound of claim 22, wherein R₃ and R₄ together with the atoms to which they are attached form a 5- to 8-membered heterocycle.

26. The compound of any one of claims 19-25, wherein R₁ and R₂ are hydrogen.

27. The compound of any one of claims 19-25, wherein R₁ is C₁-C₈ alkyl and R₂ is hydrogen.

28. The compound of any one of claims 19-25, wherein R₁ is C₁-C₃ alkyl and R₂ is hydrogen.

29. The compound of any one of claims 19-28, wherein R is hydrogen.

30. The compound of any one of claims 19-28, wherein R is C₁-C₈ alkyl.

31. The compound of any one of claims 19-28, wherein R is methyl.

32. The compound of claim 19, having the formula: 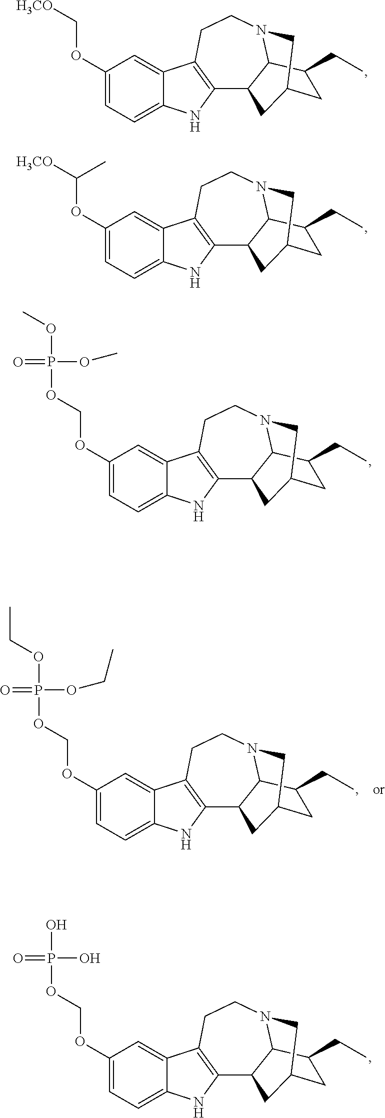 [SMILES: CC[C@H]1CC2C[C@H]3c4[nH]c5ccc(OCOC)cc5c4CC[N@@](C2)C13 ; CC[C@H]1CC2C[C@H]3c4[nH]c5ccc(OC(C)OC)cc5c4CC[N@@](C2)C13 ; CC[C@H]1CC2C[C@H]3c4[nH]c5ccc(OCOP(=O)(OC)OC)cc5c4CC[N@@](C2)C13 ; CCOP(=O)(OCC)OCOc1ccc2[nH]c3c(c2c1)CC[N@@]1CC2C[C@H](CC)C1[C@H]3C2 ; CC[C@H]1CC2C[C@H]3c4[nH]c5ccc(OCOP(=O)(O)O)cc5c4CC[N@@](C2)C13]
or a pharmaceutically acceptable salt thereof.

33. A pharmaceutical composition, comprising a compound of any one of claims 1-32 and a pharmaceutically acceptable excipient.
